(19) United States
(12) Patent Application Publication
(10) Pub. No.: US 2005/0101763 A1
(43) Pub. Date: May 12, 2005

(54) Synthesis of photolabile 2-(2-nitrophenyl)propyloxycarbonyl protected amino acids

(75) Inventors: Charles P. DeLisi, Brookline, MA (US); Richard A. Laursen, Newton, MA (US); Kumar R. Bhushan, Allston, MA (US)

Correspondence Address: RONALD I. EISENSTEIN, 100 SUMMER STREET, NIXON PEABODY LLP, BOSTON, MA, 02110, US

(73) Assignee: Trustees of Boston University, Boston, MA (US)

(21) Appl. No.: 10/954,430

(22) Filed: Sep. 30, 2004

Related U.S. Application Data
(60) Provisional application No. 60/507,365, filed on Sep. 30, 2003.

(51) Int. Cl. C07K 1/02; C07D 233/61; C07D 209/18

(52) U.S. Cl. 530/333; 548/339.1; 548/495; 560/24; 560/157; 560/159

(57) ABSTRACT

The 2-(2-nitrophenyl)propyloxycarbonyl (NPPOC) group has been introduced as a photolabile amino protecting group for amino acids to be used as building blocks in photolithographic solid-phase peptide synthesis. NPPOC-protected amino acids were found to be cleaved in the presence of UV light about twice as fast as the corresponding o-nitroveratryloxycarbonyl (NVOC)-protected amino acids. The protected amino acids are of particular use in the synthesis of peptide arrays.

### CROSS-REFERENCE TO RELATED APPLICATIONS

This application claims benefit under 35 U.S.C § 119(e) of U.S. Provisional Application No. 60/507,365, filed Sep. 30, 2003.

### GOVERNMENT SUPPORT

This work was supported by a grant from the Boston University Community Technology Fund, grant number 1928-9.

### FIELD OF THE INVENTION

The invention relates to photolabile protected amino acids and their use for the synthesis of peptide microarrays.

### BACKGROUND OF THE INVENTION

In recent years there has been interest in the synthesis of microarrays of oligonucleotides and peptides on glass or other surfaces utilizing photolithographic processes.¹ Such arrays can be used in genomics and proteomics research, respectively.¹ In 1991, Fodor et al. demonstrated that addressable arrays (e.g., peptides) could be synthesized on glass surfaces using building blocks with photolabile protecting groups. However, efforts tended to shift to oligonucleotide arrays³ because of interest in genomics analysis and the relative ease of oligonucleotide synthesis (e.g., oligonucleotide synthesis requires only four building blocks, whereas peptide synthesis requires twenty). Now, however, with the burgeoning growth of proteomics,⁴ attention is returning to peptide arrays.

In the work by Fodor and coworkers^{2,5} amino acids were protected with the photolabile o-nitroveratryloxycarbonyl (NVOC) group, which was originally introduced by Patchornik et al. in 1970.⁶ However, the photolytic removal of NVOC is not very efficient, resulting in synthesis of low quality peptides. Some improvement in the yield of photodeprotection has been reported by Holmes et al.,⁷ through use of the α-methyl-o-nitropiperonyloxycarbonyl (MeNPOC) group.

Additionally, the photodegradation products of the NVOC and MeNPOC groups include carbonyl compounds⁹ which can react with amino groups and reduce stepwise synthetic yields.

Given the growth of proteomics, as well as the scientific and commercial potential of peptide arrays, there is a need in the art to discover additional means by which quality peptides can be synthesized.

### SUMMARY OF THE INVENTION

The present invention discloses the use of a class of amino acid derivatives that contain the photoliable amino-protecting group 2-(2-nitrophenyl)propyloxycarbonyl (NPPOC), and derivatives thereof. The resulting NPPOC-protected amino acids provide improved amino acid building blocks for efficient synthesis of peptides using photo-deprotection. Methods for production of synthetic peptide microarrays using NPPOC protected amino acids are also disclosed.

In one embodiment of the invention NPPOC-protected amino acids of the general formula I are disclosed. 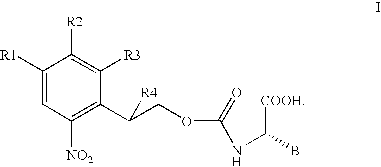
wherein
R¹=H, NO₂, CN, OCH₃, halogen, or alkyl, akoxyalkyl having 1 to 4 carbon atoms;
R²=H or OCH₃;
R³=H, F, Cl, Br or NO₂;
R¹+R² or R²+R³ can form a ring structure;
R⁴=H, halogen, OCH₃, or an alkyl radical having 1 to 4 C atoms;
B=the side chain of any amino acid.
   The amino acids that are protected with NPPOC can be natural or unnatural amino acids, e.g. L isomer, D-isomer or synthetic amino acids. Preferably the amino acid is one of the naturally occurring amino acids, for example either glycine, alanine, valine, leucine, isoleucine, serine, threonine, aspartic acid, asparagine, lysine, glutamic acid, glutamine, arginine, histidine, phenylalanine, cytosine, tryptophan, tyrosine, methionine, or proline.

In one embodiment the amino acid derivative of general formula I has a H in the R¹, R², and R³ position.

In one preferred embodiment, either R1+R2 or R2+R3 form a ring structure. That ring structure can be, for example, either alicyclic, aromatic or heterocyclic. In addition, the ring can be substituted or unsubstituted.

In another embodiment, a method for synthesizing the NPPOC protected amino acids is provided. The method comprises (a) forming 2-(2-nitrophenyl)propanol by reacting 2-ethylnitrobenzene with paraformaldehyde; (b) reacting 2-(2-nitrophenyl)propanol formed in step (a) with phosgene, or a phosgene derivative, to generate 2-(2-nitrophenyl)propyloxycarbonyl chloride; and, (c) reacting 2-(2-nitrophenyl)propyloxycarbonyl chloride of step (c) with an amino acid to generate an amino acid with NPPOC covalently attached to the amino group of the amino acid.

In still another embodiment, a method for synthesizing polypeptides in solution or on a solid phase support is provided that comprises covalently adding an amino acid to a polypeptide chain where the NPPOC-protected amino acids of claim 1 is substituted for the traditional protected amino acids of the art of polypeptide synthesis and is combined with the deprotecting of said NPPOC protecting groups with light of the appropriate wavelength.

Another embodiment of the present invention is directed to methods of synthesizing polypeptides on a solid support using the NPPOC-protected amino acids in order to make microarrays of peptides. One such method uses the photolithographic synthesis of peptides on surfaces combined with virtual masking.

### BRIEF DESCRIPTION OF DRAWINGS

FIG. 1 shows a table of reaction times, yields and masses of protected amino acid products.

FIG. 2 shows Scheme 1 that illustrates the synthesis of NPPOC chloride and its reaction with various amino acids. For synthesis, the following reagents and conditions were used: Step (a) (HCHO)ₙ, Triton B (40% in MeOH), reflux, 6 h; Step (b) COCl₂, THF, 0° C., 3 h; Step (c) Na₂CO₃, 1,4-dioxane/water (1:1), rt, ˜20 h, (Table 1).

FIG. 3 shows Scheme 2 that illustrates photolysis or deprotection of NPPOC-amino acids

FIG. 4 is a derivatization and synthesis of peptides on a glass surface. A linker, such as NPPOC-aminocaproic acid, is added in step 2. Abbreviations: HOBT, hydroxybenzotriazole; NMM, N-methylmorpholine; TBTU, O-(7-benzotriazol-1-yl)-1, 1,3,3-tetramethyluronium tetrafluoroborate; NPPOC, 2-(2-nitrophenyl)propyloxycarbonyl photolabile protecting group.

### DETAILED DESCRIPTION OF THE INVENTION

The present invention discloses using amino acid derivatives having the photolabile protective group 2-(2-nitrophenyl)propyloxycarbonyl (NPPOC), and derivatives thereof, on the amino acid. The NPPOC protected amino acids are cleaved in the presence of UV light about twice as fast as the corresponding o-nitroveratryloxycarbonyl (NVOC)-protected amino acids. Thus, the NPPOC protected amino acids are particularly useful as building blocks for peptide synthesis and can be used, for example, in photolithographic synthesis of peptides on surfaces, such as glass, membranes, filter, chips, or slides. Accordingly, the NPPOC protected amino acids provide for a means by which high density synthetic peptide arrays can be produced quickly and efficiently. Beier and Hoheisel⁸ demonstrated that the efficiency of photolytic cleavage of 2-(2-nitrophenyl)propyloxycarbonyl (NPPOC) protected nucleotides is significantly better than that for MeNPOC protected nucleotides. Another difference between the NPPOC group and the NVOC and MeNPOC groups is that the former is a derivative of 2-(2-nitrophenyl)ethyl alcohol, whereas the latter two derive from 2-nitrobenzyl alcohol. Accordingly, the additional methylene group in the NPPOC group leads to a different photocleavage mechanism.¹⁰.

As used herein, the term “peptide” refers to a polymer in which the constituent monomers are amino acids residues joined together through amide bonds. Peptides are sometimes referred to as polypeptides. As used herein, the term peptide also encompasses polypeptides that include L-optical or D-optical isomers of α-, β-, or ω-amino acids. In addition, the peptide may include amino acids having unnatural side chains or other deviations from the naturally occurring amino acids.

The term “amino acid” refers to the 20 naturally occurring amino acids or L-optical or D-optical isomers of α-, β-, or ω-amino acids. The term “amino acid” also encompasses synthetic derivatives of amino acids which may have unnatural side chains or other deviations from the naturally occurring amino acids. Preferably, the amino acid is an L-optical amino acid.

The term “receptor” refers to a molecule that has an affinity for a given ligand. Receptors may be naturally-occurring or synthetic molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells, or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex.

The term “protecting group” refers to a molecule that is chemically bound to a reactant functional group and which may be removed upon selective exposure to an activator such as electromagnetic radiation. A protecting group prevents the protected functional group from undergoing undesired side reactions. For example, when the amino group of an amino acid, such as glycine, is coupled to a the NPPOC protecting group, the protecting group prevents the amino acid from reacting during a coupling reaction between the glycine carboxylic terminus and the amino terminus of growing peptide.

The term “linker” refers to a molecule or group of molecules attached to a substrate and spacing a synthesized polypeptide from the substrate for exposure/binding to a receptor.

Synthesis of NPPOC Protected Amino Acids

The synthesis of the NPPOC protected amino acids of the present invention can be performed by any means known to those skilled in the art. In a preferred embodiment, 2-(2-nitrophenyl)propanol, or derivative thereof, is reacted with phosgene to produce an activated 2-(2-nitrophenyl)propoxycarbonyl chloride derivative of the NPPOC protecting group. The active derivative is then preferably coupled to the amino nitrogen of a natural or unnatural amino acid using standard methods, for example in the presence of sodium carbonate pH 9.5-10. (See Example 1).

After synthesis, the purity of the protected amino acid products is preferably assessed by standard means, such as ¹HNMR, CI-MS, and LC-ESI MS.

The following formula I represents the general formula for NPPOC-protected amino acids of the invention. 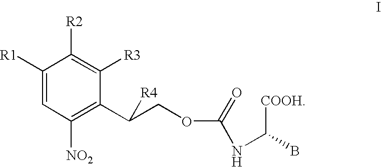
wherein
R¹=H, NO₂, CN, OCH₃, halogen, or alkyl, akoxyalkyl having 1 to 4 carbon atoms;
R²=H or OCH₃;
R³=H, F, Cl, Br or NO₂;
R¹+R² or R²+R³ can form a ring structure;
R⁴=H, halogen, OCH₃, or an alkyl radical having 1 to 4 C atoms;
B=the side chain of any amino acid.

In one preferred embodiment, the amino acid derivative of general formula I has a H in the R¹, R², and R³ position.

In one preferred embodiment, either R1+R2 or R2+R3 form a ring structure. That ring structure can be, for example, either alicyclic, aromatic or heterocyclic. In addition, the ring can be substituted or unsubstituted.

Synthesis of Ordered Peptides on an Array

The NPPOC protected amino acids of the present invention can be used in any situation where one wants to protect an amino acid side group. One preferred use is in the synthesis of polypeptides. Polypeptides can be synthesized in solution or on a surface of a solid phase support. Synthesis of polypeptides can be performed by any standard means known in the art.

In a preferred embodiment, the NPPOC protected amino acids of the present invention are used in the synthesis of peptide arrays on solid substrate surfaces. Preferably, the peptide arrays are produced using photolithographic techniques. With lithographic techniques it is possible to direct light to relatively small and precisely known locations on the substrate. Thus, it is possible to synthesize polymers of known chemical sequence at known locations of the substrate. Several photolithographic techniques useful in the present invention are described in U.S. Pat. Nos. 6,420,169, 6,416,952, 6,346,413 and 5,405,783, which are herein incorporated by reference in their entireties.

The array can be made of any conventional substrate with a surface. Moreover, the array can be in any shape that can be read, including rectangular and spheroid. Preferred substrates are any suitable rigid or semi-rigid support including glass, membranes, filter, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the peptides are bound. Preferably, the substrates are optically transparent. Any type of substrate will be a suitable “chip” as long as the peptides can be used as bait to screen for specific binders.

Any technique for production of peptide arrays known to those skilled in the art can be used to make the peptide arrays of the present invention. Since the first demonstration nearly 10 years ago by Fodor² of the principle of “light-directed, spatially addressable parallel chemical synthesis,” i.e., “synthesis on a chip,” there have been many advances in microarray technology. Although Fodor's original work described synthesis of peptide arrays, subsequent efforts have focused primarily on oligonucleotide arrays. Nevertheless, the technology for making peptide arrays exists and much of what has been learned about oligonucleotide arrays can be applied to peptides.

One of the problems with making arrays is the need for large numbers of photolithographic masks that permit selective deblocking of protected oligomers using UV light. The problem is severe in oligonucleotide synthesis where one needs four masks (corresponding to the four nucleotide bases) per synthetic cycle, but is much worse with peptides, where standard procedures would require 20 masks per cycle. To avoid this problem, a “maskless” microarray fabrication using a micromirror array such as described by Singh-Gasson¹² can be used.

In one embodiment, the arrays of the present invention are made using a glass substrate. As an example, the first step in preparation of a glass substrate array is derivatization of a glass surface with an appropriate alkoxysilane to give a surface coated with amino groups, each of which bears a NPPOC photolabile protecting group. Specific areas (pixels) on the surface are deprotected by irradiation with UV light, which is directed to these areas by the micromirror assembly, and all the exposed amino groups are then acylated by an amino acid containing a photolabile protective group. In 19 subsequent steps, all of the remaining pixels are deprotected and acylated with the 19 remaining amino acids. This marks the end of the first synthetic cycle. The process is repeated until peptides of the desired length are obtained.

A preferred reagent for introduction of functionality onto glass surfaces for many years has been aminopropyltriethoxysilane and derivatives thereof.

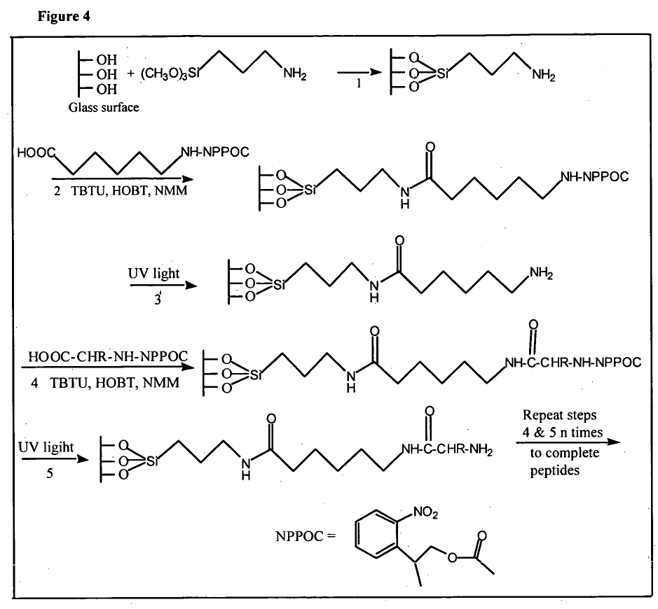

One embodiment of the present invention adapts the procedure described in Holmes^{5a}, namely silylyation with a 1:10 mixture of aminopropyltriethoxysilane: methyltriethoxysilane (the latter added to reduce the density of amino groups by a factor of 10), followed by the addition of an aminocaproic acid linker containing a photolabile protective group (FIG. 4). Any linker known in the art can be used in making the arrays of the present invention, such linkers can further contain any photolabile protecting group known to those in the art, such as NVOC or MeNPOC. Alternatively, NPPOC can be attached to the linker. Activation during coupling steps can be done, preferably, using TBTU, a standard activating agent in peptide synthesis.

In another embodiment of the present invention, an aminocaproic acid linker with a longer or more hydrophilic (e.g., polyethylene glycol) linker can be substituted, if appropriate. Thus, in one embodiment of the invention, peptides of preferably 5-20 mer (i.e., N=5-20), more preferably, 8-10 mer peptides are synthesized, as epitope mapping studies indicate that typical epitopes recognized by antibodies contain only about 6 amino acids. Because the number of different peptide sequences on a chip will be no more than several hundred thousand, only a very small fraction of all possible sixmers will be synthesized.

Protection and Deprotection of Amino Acids

The NPPOC protecting group can be removed by irradiation in the near UV or visible portion of the spectrum. The photolithographic techniques can selectively deprotect small, defined areas (pixels) on the glass surface. Deprotection thus requires efficient chemistry and engineering (i.e., the micromirror technology discussed by Singh-Gasson¹²) Preferably, NPPOC groups are removed by irradiation at 365 nm. Low wavelength light should be avoided to prevent destruction of certain amino acids, such as tryptophan. The NPPOC protecting group can be removed by any means known to those in the art for removing photlabile protective groups. For example, U.S. Pat. No. 6,552,182 describes a method for deprotecting immobilized nucleoside derivatives, especially in the production of DNA chips, which can be suitably modified for use in the present invention.

It is an important aspect that the length of time required to deprotect amino groups on a pixel be optimal. Among the preferred embodiments is the strategy described by McGall^{10a} for DNA arrays. The maskless array synthesizer (MAS)¹² is programmed to irradiate specific pixels or groups of pixels for varying periods of time, generating a gradient of partially to fully deprotected pixels. The glass substrate is then treated with any fluorescent reagent, for example, fluorescein isothiocyahate (FITC), and then visualized under the UV light. In such a way, the minimum time required for complete removal of the NPPOC group can be determined.

The NPPOC-protected amino acids of the present invention provide a means by which quality peptide arrays can be efficiently produced. Given the growing interest in proteomics, such arrays are of extreme commercial value.

The peptide arrays generated by methods of the present invention can be used for a variety of purposes, for example to screen samples of interest for molecules that bind the peptides. Samples include but are not limited to, biological samples such as, blood, urine, saliva, phlegm, gastric juices, etc., cultured cells, tissue biopsies, or other tissue preparations. It is preferred that either the target molecule or peptides are labeled with one or more labeling moieties to allow detection of peptide-molecule complexes and by comparison the lack of such a complex in the comparison sample. The labeling moieties can include compositions that can be detected by photochemical, spectroscopic, biochemical, immunochemical, chemical, optical, electrical, bioelectronic, etc. means. Labeling moieties include chemiluminescent compounds, radioisotopes, labeled compounds, spectroscopic markers such as fluorescent molecules, magnetic labels, mass spectrometry tags, electron transfer donors and/or acceptors, etc.

The arrays described herein can further be used to screen a large number of peptides for biological activity, for example by using a combinatorial peptide array. To screen for biological activity, the peptides are exposed to one or more receptors such as an antibody, whole cells, receptors on vesicles, lipids or any one of a variety of other receptors. The receptors are preferably labeled with, for example, a fluorescent marker, radiolabel, or labeled antibody reactive with the receptor and the location of the marker bound to the peptide array is detected with photon detection techniques or autoradiographic methods. Through the knowledge of the sequence of the peptide at the location where binding is detected, it is possible to determine which sequence binds with the receptor and, thus, the technique can be used to screen a large number of peptides.

Additional applications of the arrays described herein include their use as diagnostics of disease or stage of disease. In one aspect various polypeptides that bind particular receptors, such as biomarkers, would be synthesized on a substrate and screened for binding to the biomarker. In this manner, for example blood sera can be screened for the presence or absence of the biomarkers. Alternatively malignant vs. non-malignant, or diseased vs. non-diseased cell samples can be screened for receptors that are indicators of disease, or stage of disease.

The arrays of the present invention can also be used to screen antibody libraries, such as a large combinatorially generated library of antibodies that specifically bind to the peptides. Preferably, the antibodies bind to the peptides in a conformation that approximates their native state (i.e., when they are part of the protein). In this way a large library of antibodies that will bind specific native proteins is obtained.

Thus, the peptide arrays generated by means described herein have a wide variety of uses. Merely by way of example, they can be used to determine peptide sequences that bind to proteins, find sequence-specific binding drugs, identifying epitopes recognized by antibodies, and evaluation of a variety of drugs for clinical and diagnostic applications.

The invention will now be further illustrated with reference to the following examples. It will be appreciated that what follows is by way of example only and that modifications to detail may be made while still falling within the scope of the invention.

### EXAMPLES

The following example describes the synthesis of several NPPOC-amino acids.

1. Synthesis of NPPOC-Protected Amino Acids

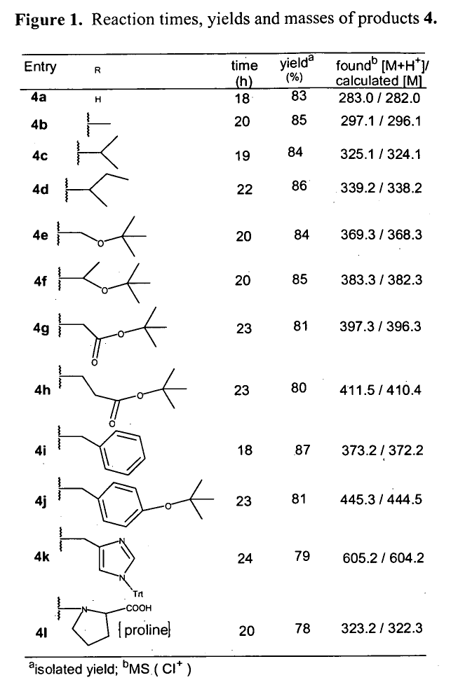

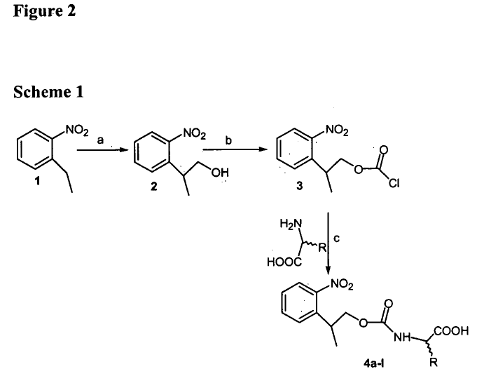

To obtain NPPOC-protected amino acids 4a-1 (see FIG. 1, Table 1), we first devised an improved synthesis of 2-(2-nitrophenyl)propanol 2 (of FIG. 2, scheme 1), based on the method of Tsuji et al.¹¹ for preparation of 2-nitrophenethyl alcohol. Triton B (40% in MeOH, 8 mmol) was added to 2-ethylnitrobenzene (8 mmol) and paraformaldehyde (8.1 mmol), and the mixture was heated at reflux for 6 h. After concentration under vacuum, the reaction mixture was neutralized using 5% aqueous HCl. The mixture was extracted with ethyl acetate (3×10 mL), dried over Na₂SO₄ and concentrated at reduced pressure. The residue was purified by flash chromatography using hexane-ethyl acetate (4:1) to give compound 2 (96%, red oil). ¹H NMR (CDCl₃, 400 MHz): δ/ppm 7.73 (d, J=8.0 Hz, 1H, Ar—H), 7.56 (t, J=7.4 Hz, 1H, Ar—H), 7.48 (d, J=7.6 Hz, 1H, Ar—H), 7.35 (t, J=7.6 Hz, 1H, Ar—H), 3.77 (d, J=6.4 Hz, 2H, CH₂), 3.51 (m, 1H, CH), 1.79 (br s, 1H, OH), 1.32 (d, J=6.8 Hz, 3H, CH3); MS (Cl+) m/z: 182.1 (M+H+).

The alcohol 2 (of FIG. 2, scheme 1) was then treated with phosgene to give NPPOC chloride 3 (of FIG. 2, scheme 1). A solution of 2 (6 mmol) in anhydrous THF (5 mL) at 0° C., was added a solution of phosgene (20% in toluene, 9 mmol) over a period of 15 min with stirring under nitrogen atmosphere. After 45 min, the ice bath was removed and stirring was continued at room temperature for 2 h. A stream of N₂ was then bubbled through the solution for 1 h to remove the excess phosgene, after which the mixture was evaporated to dryness under vacuum to give compound 3 (99%, brown oil). ¹H NMR (CDCl₃, 400 MHz): δ/ppm 7.81 (d, J=8.0 Hz, 1H, Ar—H), 7.60 (t, J=7.4 Hz, 1H, Ar—H), 7.43 (d, J=7.6 Hz, 1H, Ar—H), 7.38 (t, J=7.6 Hz, 1H, Ar—H), 4.47 (d, J=6.4 Hz, 2H, CH₂), 3.77 (m, 1H, CH), 1.39 (d, J=6.8 Hz, 3H, CH₃); MS (Cl+) m/z: 243.6 (M+H+).

Reaction of 3 (of FIG. 2, scheme 1) with various amino acids in the presence of sodium carbonate (pH 9.5-10; reaction in sodium bicarbonate gave some dipeptide material) generated 4a-1. The products 4a-1 and their purity were assessed by ¹H NMR, CI-MS and LC-ESI-MS. Na₂CO₃ (2.2 mmol) was added to the solution of L-amino acid (1 mmol) in 10 mL water/1,4-dioxane (1:1) at 0° C., followed by the dropwise addition of 3 (1 mmol, in 1 mL THF). After 20 min the ice bath was removed and stirring was continued for 18-24 h. The reaction mixture was evaporated to dryness, 3 mL of water was added and the mixture was extracted with ethyl acetate (2×5 mL) to remove 3 or its hydrolysis product. The aqueous layer was acidified by addition of 5% HCl at 0° C. and extracted with ethyl acetate (3×10 mL); the extracts were dried over Na₂SO₄ and concentrated at reduced pressure to give a glassy substance that, in most cases was essentially pure (free of by-products), based on spectroscopic measurements.

The Spectroscopic data for selected products follows: 4b: ¹H NMR (CDCl₃, 400 MHz): δ/ppm 7.71 (d, J=8.0 Hz, 1H, Ar—H), 7.54 (t, J=7.4 Hz, 1H, Ar—H), 7.43 (d, J=7.6 Hz, 1H, Ar—H), 7.34 (t, J=7.6 Hz, 1H, Ar—H), 5.28 (br d, 1H, NH), 4.28 (d, J=6.4 Hz, 2H, CH₂), 4.11 (m, 1H, CH), 3.67 (m, 1H, CH), 1.40 (d, J=7.6 Hz, 3H, CH₃), 1.31 (d, J=6.8 Hz, 3H, CH₃); LC-MS (ESI+) m/z: 297.1 (M+H+), 319.1 (M+Na+).
4e: ¹H NMR (CDCl₃, 400 MHz): δ/ppm 7.70 (d, J=8.0 Hz, 1H, Ar—H), 7.52 (t, J=7.4 Hz, 1H, Ar—H), 7.42 (d, J=7.6 Hz, 1H, Ar—H), 7.32 (t, J=7.6 Hz, 1H, Ar—H), 5.42 (br d, 1H, NH), 4.25 (d, J=6.4 Hz, 2H, CH₂), 4.09 (m, 1H, CH), 3.98 (m, 1H, CH₂), 3.82 (m, 1H, CH₂), 3.53 (m, 1H, CH), 1.28 (d, J=6.8 Hz, 3H, CH₃), 1.10 (s, 9H, 3×CH₃); LC-MS (ESI⁺) m/z: 369.1 (M+H⁺), 391.1 (M+Na⁺).
4h: ¹H NMR (CDCl₃, 400 MHz): δ/ppm 7.67 (d, J=8.0 Hz, 1H, Ar—H), 7.50 (t, J=7.4 Hz, 1H, Ar—H), 7.41 (d, J=7.6 Hz, 1H, Ar—H), 7.30 (t, J=7.6 Hz, 1H, Ar—H), 5.48 (br d, 1H, NH), 4.23 (d, J=6.4 Hz, 2H, CH₂), 4.16 (m, 1H, CH), 3.62 (m, 1H, CH), 2.28 (m, 2H, CH₂), 2.09 (m, 1H, CH₂), 1.90 (m, 1H, CH₂), 1.37 (s, 9H, 3×CH₃), 1.31 (d, J=6.8 Hz, 3H, CH₃); LC-MS (ESI⁺) m/z: 411.1 (M+H⁺), 433.1 (M+Na⁺).
4j: ¹H NMR (CDCl₃, 400 MHz): δ/ppm 7.70 (d, J=8.0 Hz, 1H, Ar—H), 7.51 (t, J=7.4 Hz, 1H, Ar—H), 7.40 (d, J=7.6 Hz, 1H, Ar—H), 7.29 (t, J=7.6 Hz, 1H, Ar—H), 6.98 (d, J=8.2 Hz, 2H, Ar—H), 6.87 (d, J=8.2 Hz, 2H, Ar—H) 5.10 (br d, 1H, NH), 4.24 (d, J=6.4 Hz, 2H, CH₂), 4.16 (m, 1H, CH), 3.65 (m, 1H, CH₂), 3.06 (m, 1H, CH₂), 2.98 (m, 1H, CH₂), 1.30 (s, 9H, 3×CH₃), 1.26 (d, J=6.8 Hz, 3H, CH₃); LC-MS (ESI⁺) m/z: 445.2 (M+H⁺), 467.2 (M+Na⁺).
4l: ¹H NMR (CDCl₃, 400 MHz): δ/ppm 7.71 (d, J=8.0 Hz, 1H, Ar—H), 7.52 (t, J=7.4 Hz, 1H, Ar—H), 7.41 (d, J=7.6 Hz, 1H, Ar—H), 7.32 (t, J=7.6 Hz, 1H, Ar—H), 4.29 (d, J=6.4 Hz, 2H, CH₂), 4.15 (m, 1H, CH), 3.65 (m, 2H, CH₂), 3.46 (m, 1H, CH), 1.80-2.20 (m, 4H, 2×CH₂), 1.29 (d, J=6.8 Hz, 3H, CH₃); LC-MS (ESI⁺) m/z: 323.1 (M+H⁺), 345.1 (M+Na⁺).

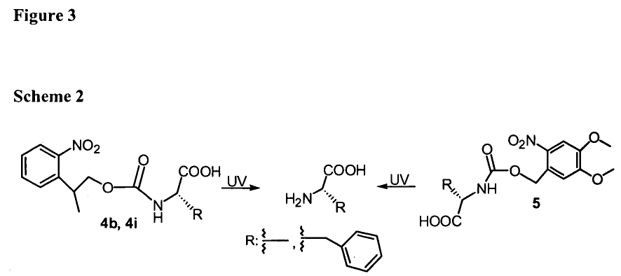

Rates of photolysis of NPPOC-amino acids 4b, 4i (see FIG. 3, Scheme 2) were compared with those of the corresponding NVOC-amino acids 5 in solution under identical conditions (irradiation at 365 nm, 2000 μW/cm², 5 mM solvent) (FIG. 3, scheme 2). Of the several solvent conditions tested for the photodeprotection in these preliminary studies, acidic methanol (2.5 mM semicarbazide hydrochloride in methanol) gave the best results. LC-MS analysis indicated that the NPPOC derivatives were cleaved about twice as fast as the corresponding NVOC derivatives. The solvents used for photodeprotection of 4a, 4b, 4c, 4d, and 4f (see table 1) were, (a) 1,4-dioxane; (b) acetonitrile; (c) methanol; (d) 2.5 mM diisopropylethylamine in methanol; (f) 2.5 mM semicarbazide hydrochloride in methanol.

In conclusion, we have developed an efficient method for the synthesis of photolabile 2-(2-nitrophenyl)propyloxycarbonyl (NPPOC) protected amino acids for use as building blocks for photolithographic solid-phase peptide synthesis. These derivatives undergo light-promoted deprotection at a rate at least twice that of the earlier described⁵ NVOC amino acids, due, presumably, to different cleavage mechanisms.

### REFERENCES

The references cited herein and throughout the specification are incorporated by reference.
1. (a) Housemen, T. B.; Huh, J. H.; Kron, S. J.; Mrksich, M. Nat. Biotechnol., 2002, 20, 270; (b) Vernet, G. Virus Res., 2002, 82, 65; (C) Emili, A. Q.; Cagney, G. Nat. Biotechnol., 2000, 18, 393.
2. Fodor, S. P. A.; Read, J. L.; Pirrung, M. C.; Stryer, L.; Lu, A. T.; Solas, D. Science, 1991, 251, 767.
3. (a) Pease, A. C.; Solas, D.; Sullivan, E. J.; Cronin, M. T.; Holmens, C. P.; Fodor, S. P. A. Proc. Natl. Acad. Sci. (USA), 1994, 91, 5022; (b) Niemeyer, C. M.; Blohm, D. Angew. Chem. Int. Ed., 1999, 38, 2865; (c) Singh-Gasson, S.; Green, R. D.; Yue, Y.; Nelson, C.; Blattner, F.; Sussman, M. R.; Cerrina, F. Nat. Biotechnol., 1999, 17, 974.; (d) Frank, R. Comb. Chem. High Throughput Screen., 2002, 5, 429
4. Robinson, W. H.; Steinman, L.; Utz, P. J. Arthritis & Rheumatism, 2002, 46, 885
5. Holmes, C. P.; Adams, C. L.; Kochersperger, L. M.; Mortensen, R. B.; Aldwin, L. A. Biopolymers, 1995, 37, 199; (b) Read, J. L.; Fodor, S. P. A.; Stryer, L.; Pirrung, Michael C.; Hoeprich, P. D., 2002, U.S. Pat. No. 6,420,169; (c) Pirrung, M. C.; Stryer, L.; Fodor, S. P. A.; Read, J. L., 2002, U.S. Pat. No. 6,416,952.
6. (a) Patchornik, A.; Amit, B.; Woodward, R. B. J. Am. Chem. Soc., 1970, 92, 6333; (b) Pillai, V. N. R. Synthesis, 1980, 1.
7. Holmes, C. P.; Solas, D. W.; Kiangsoontra, B. PCT Int. Appl., 1994, WO 9410128.
(a) Beier, M.; Hoheisel, J. D. Nucleic Acids Res., 2000, 28, el 1; (b) Beier, M.; Hoheisel, J. D. Nucleic Acids Res., 1999, 27, 1970.
9. (a) McGall, G. H; Barone, A. D.; Diggelmann, M.; Fodor, S. P. A.; Gentalen, E.; Ngo, N. J. Am. Chem. Soc., 1997, 119, 5081; (b) Bochet, C. G. Tetrahedron Lett., 2000, 41, 6341;
10. Hasan, A.; Stengele, K.; Giegrich, H.; Cornwell, P.; Isham, K. R.; Sachleben, R. A.; Pfleiderer, W. Foote, R. S. Tetrahedron, 1997, 53, 4247;
11. Tsuji, Y.; Kotachi, S.; Huh, K. T.; Watanabe, Y. J. Org. Chem. 1990, 55, 580.
12. Singh-Gasson, S., Green, R. D., Yue, Y., Nelson, C. Blattner, F. Sussman, M. R. and Cerrina, F., Nat. Biotechnol. 1999 17, 974

1. A protected amino acid residue, wherein the amino acid has a photolabile protective group of the general formula I, 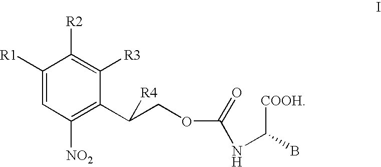
wherein
R¹=H, NO₂, CN, OCH₃, halogen, or alkyl, akoxyalkyl having 1 to 4 carbon atoms;
R²=H or OCH₃;
R³=H, F, Cl, Br or NO₂;
R¹+R² or R²+R³ can form a ring structure;
R⁴=H, halogen, OCH₃, or an alkyl radical having: 1 to 4 C atoms;
B=the side chain of any amino acid.

2. The protected amino acid residue of claim 1, wherein R¹, R², and R³ are H.

3. The protected amino acid residue of claim 1, wherein said R¹+R² or R²+R³ ring structure is alicyclic.

4. The protected amino acid residue of claim 1, wherein said R¹+R² or R²+R³ ring structure is aromatic.

5. The protected amino acid residue of claim 1, wherein said R¹+R² or R²+R³ ring structure is heterocyclic.

6. The protected amino acid residue of claim 1, wherein the amino acid is selected from the group consisting of naturally occurring amino acids glycine, alanine, valine, leucine, isoleucine, serine, threonine, aspartic acid, asparagine, lysine, glutamic acid, glutamine, arginine, histidine, phenylalanine, cytosine, tryptophan, tyrosine, methionine, or proline.

7. The protected amino acid residue of claim 1, wherein the amino acid is a synthetic amino acid.

8. A method for preparing the protected amino acid residue of claim 1 comprising:
(a) forming 2-(2-nitrophenyl)propanol by reacting 2-ethylnitobenzene with paraformaldehyde;
(b) reacting 2-(2-nitrophenyl)propanol formed in step (a) with phosgene, or a phosgene derivative, to generate 2-(2-nitrophenyl)propyloxycarbonyl chloride; and
(c) reacting 2-(2-nitrophenyl)propyloxycarbonyl chloride of step (c) with an amino acid to generate an amino acid with NPPOC covalently attached to the amino group of the amino acid.

9. A method for synthesizing polypeptides in solution or on a solid phase support comprising covalently adding a protected amino acid residue of claim 1 to a polypeptide chain, and repeating until the desired protected polypeptide sequence is formed.

10. The method of claim 10, further comprising deprotecting the polypeptide by exposing said NPPOC protecting groups to light of the desired wavelength.

11. A method for making an array of polypeptides comprising synthesizing peptides on a solid support wherein the peptides are synthesized using the protected amino acid residues of claim 1.
